(19) United States
(12) Patent Application Publication
(10) Pub. No.: US 2024/0257971 A1
(43) Pub. Date: Aug. 1, 2024

(54) VIRTUAL REALITY NEUROPSYCHOLOGICAL ASSESSMENT

(71) Applicant: Arizona Board of Regents on behalf of the University of Arizona, Tucson, AZ (US)

(72) Inventors: William Killgore, Tucson, AZ (US); Janet Roveda, Tucson, AZ (US); Jerzy Rozenblit, Tucson, AZ (US); Ao Li, Tucson, AZ (US); Huayu Li, Tucson, AZ (US)

(21) Appl. No.: 18/430,445

(22) Filed: Feb. 1, 2024

Related U.S. Application Data
(60) Provisional application No. 63/442,674, filed on Feb. 1, 2023.

(51) Int. Cl. G16H 50/20

(52) U.S. Cl. CPC G16H 50/20

(57) ABSTRACT

A virtual reality neuropsychological assessment (VRNA) system uses a deep learning network and a VR headset to administer multi-domain assessments of human cognitive performance. The deep learning network is trained to identify features in sensor data indicative of neuropsychological performance and classify users based on the features identified in the sensor data. The VR headset provides a user with a virtual simulation of an activity involving decision-making scenarios. During the virtual simulation, sensor data via a plurality of sensors of the VR headset is captured. The sensor data is applied to the deep learning network to identify features of the user and classify the user based on the features into a neuropsychological domains, such as attention, memory, processing speed, and executive function. Sensor data includes eye-tracking, hand-eye motor coordination, reaction time, working memory, learning and delayed memory, and inhibitory control.

### CROSS-REFERENCE TO RELATED APPLICATIONS

This application claims the benefit of U.S. Provisional 63/442,674 filed Feb. 1, 2023, which is hereby incorporated by reference as if submitted in its entirety.

### STATEMENT REGARDING FEDERALLY SPONSORED RESEARCH OR DEVELOPMENT

This invention was made with government support under Grant #HT94252310877 awarded by the US Army Medical Research Acquisition Activity. The government has certain rights in the invention.

### BACKGROUND

During the past 20 years, there have been more than 414,000 traumatic brain injuries (TBIs) reported among military personnel, with the vast majority of these (˜83%) being categorized as mild traumatic brain injuries (mTBI). Cognitive deficits from mTBI often include problems with attention, memory, processing speed, and executive functioning, all of which can have downstream effects on other complex cognitive abilities 1-3. It is therefore of critical importance to provide military and civilian medical personnel with advanced technologies to accurately determine the extent of injuries and provide automated decision guidance regarding return-to-duty, temporary rest, or evacuation (if possible). The gold standard for determining cognitive deficits from an injury is a traditional clinician administered neuropsychological assessment battery. Such batteries were designed to comprehensively evaluate a spectrum of cognitive abilities and functional domains and, historically, were measured using specialized paper-and-pencil tests that require administration, scoring, and interpretation by a trained psychologist, often taking as long as six to eight hours to administer. Thus, traditional neuropsychological assessments are impractical and inappropriate for use in far-forward military settings. There is a critical need for portable, automated, and ecologically valid tools which can use advanced sensor technology and machine learning to rapidly assess neurocognitive deficits due to brain injury in austere environments.

### BRIEF SUMMARY OF THE INVENTION

The present embodiments may relate to, inter alia, systems and methods for providing a neuropsychological assessment that combines advanced sensor technology with artificial intelligence/machine learning (AI/ML) using neural networks. The neuropsychological assessment may be used to assess multiple domains of real-time cognitive performance of a participant. The neuropsychological assessment may use a brief simulation on a portable virtual reality (VR) device and provide a personalized assessment of the participant's neurocognitive status. The disclosed technology would be time-efficient by allowing an extensive assessment of a broad set of capacities in only a few minutes, while also maintaining participant interest and ecological validity of the tasks. In at least one embodiment, a real-time stream of information may be evaluated using AI/ML algorithms to extract multiple dimensions of cognitive performance simultaneously.

In one aspect, an operational neurocognitive assessment of mild traumatic brain injuries (mTBIs) is provided that capitalizes on recent advances in sensor technology, AI/ML, and computational neuroscience. With respect to the disclosed technology, a VR headset and sensor system may be used to provide a simple game-like scenario that is developed to allow multi-domain assessment of cognitive performance using AI/ML algorithms. Additionally, or alternatively, a rugged, portable VR neuropsychological assessment system based on AI/ML and neural network learning is described herein. In some embodiments of the disclosed technology, a portable and neurocognitive assessment system is provided. The portable system may incorporate advanced VR and sensors that can be administered easily in only a few minutes, while providing valid identification of cognitive deficits at a level commensurate with (or better than) conventional neuropsychological assessment batteries. Further, a core game-like VR scenario that incorporates data collection from multiple sensors that can be assessed in real-time using AI/ML is described herein. The portable system may provide four primary virtual reality neuropsychological assessment (VRNA) domains of Attention, Memory, Executive Function, and Processing Speed, that may be extracted from responses to a brief VR game-like scenario.

In a second aspect, the following description relates to, inter alia, technical improvements to neuropsychological testing using virtual reality (VR) systems. Described herein are methods and systems directed to virtual reality neuropsychological assessments (VRNA). The disclosed technology uses virtual reality (e.g., a three-dimensional simulation of combat activity involving game-like decision-making) to assess neuropsychological performance and identify neurocognitive deficits (e.g., caused by brain injuries). In some embodiments, users may participate in a virtual combat scenario, for example where they conduct a predefined mission. The mission may include engaging enemy combatants, friendly forces, non-combatants, and various aerial and land vehicles per rules of engagement (i.e., contingencies). The rules of engagement may define when to act or not act on combatants based on stimulus characteristics and the situational context. A user's performance during the mission may be analyzed one or more trained neural networks to assess the user's cognitive function in a plurality of domains (e.g., attention, memory, executive function, and processing speed). In some embodiments of the disclosed technology, parameters of the stimuli may be parametrically modulated to require varying levels of cognitive performance. By parametrically modulating the cognitive load (i.e., task difficulty), the system can extract several dimensions of cognitive performance simultaneously.

In a third aspect of the VRNA assessment, raw sensory data may be captured during a participants' virtual activities while participating in a virtual scenario using a VR headset that provides an immersive experience. Raw sensory data (e.g., eye-tracking data and data indictive of hand-eye motor coordination, reaction time, working memory, learning and delayed memory, and inhibitory control) may be provided to a deep learning network trained to identify features indicative of neuropsychological performance, calculate scores in a number of neuropsychological domains, and classify users based on those domain scores. In some embodiments, for instance, the deep neural network may calculate scores in the four domains measured in a traditional neuropsychological test battery (e.g., attention, memory, processing speed, and executive function), which are most likely to discriminate between healthy participants and those who have suffered a mild traumatic brain injury (mTBI). Additionally, or alternatively, the deep neural network may calculate scores in other cognitive domains (e.g., language processing, visuo-spatial abilities, balance, motor functioning, perception, memory subcomponents, emotional/psychological functioning, etc.). To incorporate temporal and spatial correlations into one network, the deep learning network may employ both long short-term memory and convolution techniques. For instance, a convolutional neural network (CNN) may be trained to extract features from the signals captured during the participants' virtual activities and a recurrent neural network (RNN) architecture (e.g., long short-term memory (LSTM) network) may use a training dataset to create a sequence of temporal and spatial correlations in the deep learning network.

### BRIEF DESCRIPTION OF THE DRAWINGS

For a detailed description of various examples, reference will now be made to the accompanying drawings.

FIG. 1 shows, in block diagram form, a simplified network diagram in accordance with one or more embodiments described herein.

FIG. 2 shows, in block diagram form, a simplified system diagram for an electronic device in accordance with one or more embodiments described herein.

FIGS. 3A and 3B show exemplary flow diagrams in accordance with one or more embodiments described herein.

FIG. 4A shows an example VR headset used for a VRNA assessment in accordance with one or more embodiments described herein.

FIG. 4B shows an example view of immersive content provided by a VR headset for a VRNA assessment in accordance with one or more embodiments described herein.

FIG. 5 shows an overview diagram of VRNA assessment in accordance with one or more embodiments described herein.

FIG. 6 shows an example diagram of a neural network in accordance with one or more embodiments described herein.

FIG. 7 shows an example view of a task provided to a user via a VR headset for a VRNA assessment in accordance with one or more embodiments described herein.

FIG. 8 shows an example implementation of a VRNA assessment in accordance with one or more embodiments described herein.

FIG. 9 shows an example data analysis flow diagram in accordance with one or more embodiments described herein.

### DETAILED DESCRIPTION

The disclosed technology relates to the development of a neuropsychological assessment that combines advanced sensor technology with a type of artificial intelligence/machine learning (AI/ML) known as deep neural network learning (DNN). The DNN may be used to assess multiple domains of real-time cognitive performance using a brief simulation on a portable virtual reality (VR) device and provide personalized assessment of neurocognitive status. The disclosed approach would be time-efficient by allowing an extensive assessment of a broad set of capacities in only a few minutes, while also maintaining examinee interest and ecological validity of the tasks. In at least one embodiment, a real-time stream of information may be evaluated using DNN AI/ML algorithms to extract multiple dimensions of cognitive performance simultaneously.

While there exists a plethora of well-established clinically administered neuropsychological tests and batteries to measure cognitive and behavioral deficits associated with mild traumatic brain injuries (mTBI), the vast majority of these are excessively time intensive, cumbersome, and nearly impossible to score and interpret handily in an operational environment. There is therefore a critical need for an operational neurocognitive assessment of mTBI that capitalizes on recent advances in sensor technology, AI/ML, and computational neuroscience. With respect to the disclosed technology, a VR headset and sensor system may be used to provide a simple game-like scenario that is developed to allow multi-domain assessment of cognitive performance using DNN AI/ML algorithms. Additionally, or alternatively, a rugged, portable VR neuropsychological assessment system based on AI/ML and neural network learning is described herein. In some embodiments of the disclosed technology, a portable and neurocognitive assessment system is provided. The portable system may incorporate advanced VR and sensors, which can be administered easily in only a few minutes, while providing valid identification of cognitive deficits at a level commensurate with (or better than) conventional neuropsychological assessment batteries. Further, a core game-like VR scenario that incorporates data collection from multiple sensors that can be assessed in real-time using DNN AI/ML is described herein. The portable system may provide four primary virtual reality neuropsychological assessment (VRNA) domains of Attention, Memory, Executive Function, and Processing Speed. Domain results may be extracted from responses to a brief VR game-like scenario.

The following description relates to, inter alia, technical improvements to neuropsychological testing using virtual reality (VR) systems. Described herein are methods and systems directed to virtual reality neuropsychological assessments (VRNA). The disclosed technology uses virtual reality to assess neuropsychological performance and identify neurocognitive deficits (e.g., caused by brain injuries). In some embodiments, users may participate in a virtual combat scenario or other interactive game-like decision-making scenarios. For example, in a virtual combat scenario, a participant may be expected to conduct a predefined mission while engaging enemy combatants, friendly forces, non-combatants, and various aerial and land vehicles and rules of engagement (i.e., contingencies). The rules of engagement may define when to fire or hold-fire on combatants based on stimulus characteristics and the situational context.

In some embodiments of the disclosed technology, parameters of the stimuli may be parametrically modulated to require varying levels of cognitive performance. By parametrically modulating the cognitive load (i.e., task difficulty), the system may extract several dimensions of cognitive performance simultaneously.

In some embodiments, raw sensory data captured during the participants' virtual activities—e.g., eye-tracking data and data indictive of hand-eye motor coordination, reaction time, working memory, learning and delayed memory, and inhibitory control—may be provided to a deep learning network trained to identify features indicative of neuropsychological performance, calculate scores in a number of neuropsychological domains, and classify users based on those domain scores. In some embodiments, for instance, the deep neural network may calculate scores in the four domains measured in a traditional neuropsychological test battery (e.g., attention, memory, processing speed, and executive function), which are most likely to discriminate between healthy participants and those who have suffered a mild traumatic brain injury. Additionally, or alternatively, the deep neural network may calculate scores in other cognitive domains (e.g., language processing, visuo-spatial abilities, balance, motor functioning, perception, memory subcomponents, emotional/psychological functioning, etc.).

To incorporate temporal and spatial correlations into one network, the deep learning network may employ both long short-term memory and convolution techniques. For instance, a convolutional neural network (CNN) may be trained to extract features from the signals captured during the participants' virtual activities and a recurrent neural network (RNN) architecture (e.g., long short-term memory (LSTM) network) may use a training dataset to create a sequence of temporal and spatial correlations in the deep learning network.

Reference in this disclosure to “one embodiment” or to “an embodiment” means that a particular feature, structure, or characteristic described in connection with the embodiment is included in at least one embodiment, and multiple references to “one embodiment” or to “an embodiment” should not be understood as necessarily all referring to the same embodiment or to different embodiments.

In the following description, for purposes of explanation, numerous specific details are set forth in order to provide a thorough understanding of the disclosed concepts. As part of this description, some of this disclosure's drawings represent structures and devices in block diagram form in order to avoid obscuring the novel aspects of the disclosed embodiments. In this context, it should be understood that references to numbered drawing elements without associated identifiers (e.g., 100) refer to all instances of the drawing element with identifiers (e.g., 100a and 100b). Further, as part of this description, some of this disclosure's drawings may be provided in the form of a flow diagram. The boxes in any particular flow chart may be presented in a particular order. However, it should be understood that the particular flow of any flow diagram is used only to exemplify one embodiment. In other embodiments, any of the various components depicted in the flow chart may be deleted, or the components may be performed in a different order, or even concurrently. In addition, other embodiments may include additional steps not depicted as part of the flow chart. The language used in this disclosure has been principally selected for readability and instructional purposes and may not have been selected to delineate or circumscribe the disclosed subject matter. Reference in this disclosure to “one embodiment” or to “an embodiment” means that a particular feature, structure, or characteristic described in connection with the embodiment is included in at least one embodiment, and multiple references to “one embodiment” or to “an embodiment” should not be understood as necessarily all referring to the same embodiment or to different embodiments.

It should be appreciated that in the development of any actual implementation (as in any development project), numerous decisions must be made to achieve the developers' specific goals (e.g., compliance with system and business-related constraints), and that these goals will vary from one implementation to another. It will also be appreciated that such development efforts might be complex and time consuming but would nevertheless be a routine undertaking for those of ordinary skill in the art of virtual reality technology having the benefit of this disclosure.

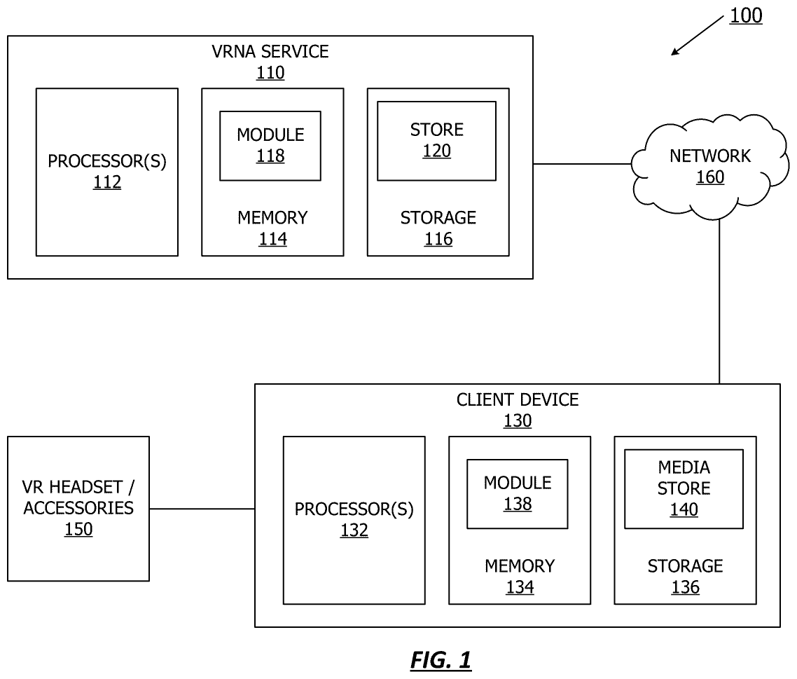

Referring to FIG. 1, a simplified block diagram 100 is depicted of a virtual reality neuropsychological assessment (VRNA) service 110 connected to a client device 130, for example over a network 160. Client device 130 may be a multifunctional device, such as a personal computer, laptop computer, tablet computer, personal digital assistant, or the like. Client device 130 may be connected to a VR headset 150, for example over a wireless connection, such as a short-range wireless network or wireless local area network (WLAN), or over a wired connection. In some embodiments, the connection between the client device 130 and VR headset 150 may require high throughput and low latency to satisfy high data throughput requirements of high-resolution video transmission. Additionally, or alternatively, the connection requirement between the client device 130 and VR headset 150 may require high throughput and low latency to satisfy data throughput requirements to provide an immersive experience so that meaningful neuropsychological outcome data can be obtained.

VRNA service 110 may include one or more servers or other computing or storage devices on which the various modules and storage devices may be contained. Although VRNA service 110 is depicted as comprising various components in an exemplary manner, in one or more embodiments, the various components and functionality may be distributed across multiple network devices, such as multiple servers, multiple network storage devices, or combinations thereof. Further, additional components may be used and some combination of the functionality of any of the components may be combined. VRNA service 110 may include one or more processors 112, one or more memory devices 114, and one or more storage devices 116. The one or more processors 112 may include one or more of a central processing unit (CPU), a graphical processing unit (GPU), or the like. Further, processor 112 may include multiple processors of the same or different type. Memory devices 114 may each include one or more different types of memory, which may be used for performing device functions in conjunction with processor 112. For example, memory 114 may include cache, ROM, and/or RAM. Memory 114 may store various programming modules during execution, including VRNA assessment module 118.

VRNA service 110 may use storage 116 to store, inter alia, media files, media file data, program data, VRNA assessment data (e.g., outcome data), AI/ML algorithms, simulation data, or the like. Additional data may include, but is not limited to, neural network training data, DNN configuration data or the like. VRNA service 110 may store this data in a media store 120 within storage 116. Storage 116 may include one or more physical storage devices. The physical storage devices may be located within a single location, distributed across multiple storages devices, such as multiple servers, or a combination thereof.

In another embodiment, store 120 may include model training data for a dataset to train a model. Model training data may include labeled training data that a machine learning model uses to learn and then be enabled to predict and identify neurocognitive deficits. In some embodiments, training data may consist of data pairs of input and output data. For example, input data may include sensory data (e.g., eye-tracking data, hand-eye motor coordination, reaction time, inhibitory control) that been processed along with a corresponding label. The label may be objective or subjective. Additionally, the label may be obtained through detailed experimentation with human users. An objective label may for example, indicate a measure obtained by computing a metric based on a traditional neuropsychological test battery (e.g., attention, memory, processing speed, and executive function). A subjective label may for example, indicate a perceptual score that is assigned by a human annotator.

Client device 130 may include one or more devices or other computing or storage devices on which the various modules and storage devices may be contained. Client device 130 may be, for example, a personal computer, a laptop, a tablet PC, or a head-mounted device. Although client device 130 is depicted as comprising various components in an exemplary manner, in one or more embodiments, the various components and functionality may be distributed across multiple network devices, such as multiple devices, multiple network storage devices, or combinations thereof. Further, additional components may be used and some combination of the functionality of any of the components may be combined. Client device 130 may include one or more processors 132, one or more memory devices 134, and one or more storage devices 136. The one or more processors 132 may include one or more of a central processing unit (CPU), a graphical processing unit (GPU), or the like. Further, processor 132 may include multiple processors of the same or different type. Memory devices 134 may each include one or more different types of memory, which may be used for performing device functions in conjunction with processor 132. For example, memory 134 may include cache, ROM, and/or RAM. Memory 134 may store various programming modules during execution, including virtual simulation module 138. In some embodiments, the virtual simulation module 130 may provide a VRNA assessment as described herein. Additionally, or alternatively, the client device may store a trained neural network to not only provide the virtual simulation but also provide results of the virtual simulation using the trained neural network.

Client device 130 may use storage 136 to store, inter alia, media files, media file data, program data, API data, simulation software, VRNA assessment data (e.g., outcome data), AI/ML algorithms, simulation data, or the like. Additional data may include, but is not limited to, neural network training data, DNN configuration data or the like. Client device 130 may store this data in a media store 140 within storage 136. Storage 136 may include one or more physical storage devices. The physical storage devices may be located within a single location, distributed across multiple storages devices, such as multiple servers, or a combination thereof.

Client device 130 may be connected to a VR headset and one or more accessories 150. The VR headset may be a head-mounted device that comprises a stereoscopic display. The VR headset, in some embodiments, may provide a user with an immersive and interactive experience, as described herein. Accessories provided may include vital monitors (e.g., heart rate monitor), game controllers, or the like. In some embodiments, the VR headset may be configured to include a virtual simulation for the VRNA assessment described herein. Additionally, or alternatively, the VR headset may be configured to include a trained neural network to not only provide the virtual simulation but also provide results of the virtual simulation using the trained neural network.

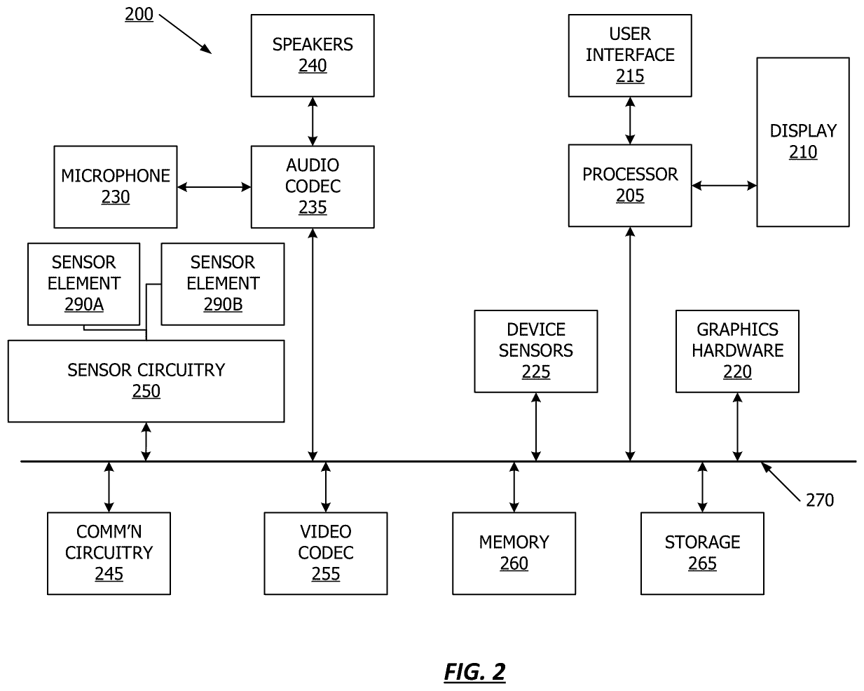

Referring now to FIG. 2, a simplified functional block diagram of illustrative multifunction device 200 is shown according to one embodiment. Multifunctional device 200 may show representative components, for example, for devices of VRNA service 110, client device 130, and VR headset 150 of FIG. 1. Multifunction electronic device 200 may include processor 205, display 210, user interface 215, graphics hardware 220, device sensors 225 (e.g., proximity sensor/ambient light sensor, accelerometer and/or gyroscope), microphone 230, audio codec(s) 235, speaker(s) 240, communications circuitry 245, digital image capture circuitry 250 (e.g., including camera system) video codec(s) 255 (e.g., in support of digital image capture unit), memory 260, storage device 265, and communications bus 270. Multifunction electronic device 200 may be, for example, a digital camera or a personal electronic device such as a personal digital assistant (PDA), personal music player, mobile telephone, a wearable device (e.g., VR headset, smart watch), a tablet computer, or the like.

Processor 205 may execute instructions necessary to carry out or control the operation of many functions performed by device 200 (e.g., such as the generation and/or processing of images as disclosed herein). Processor 205 may for instance, drive display 210 and receive user input from user interface 215. User interface 215 may allow a user to interact with device 200. For example, user interface 215 can take a variety of forms, such as a button, keypad, dial, a click wheel, keyboard, display screen and/or a touch screen. Processor 205 may also, for example, be a system-on-chip such as those found in mobile devices and include a dedicated graphics processing unit (GPU). Processor 205 may be based on reduced instruction-set computer (RISC) or complex instruction-set computer (CISC) architectures or any other suitable architecture and may include one or more processing cores. Graphics hardware 220 may be special purpose computational hardware for processing graphics and/or assisting processor 205 to process graphics information. In one embodiment, graphics hardware 220 may include a programmable GPU.

Sensor circuitry 250 may include two (or more) sensor elements 290A and 290B. Sensor elements may be temperature sensors, image sensors, heart rate monitors, or the like. Sensor circuitry 250 may capture still and/or video images, a user's body temperature, a user's heart rate, or the like. Output from sensor circuitry 250 may be processed, at least in part, by video codec(s) 255 and/or processor 205 and/or graphics hardware 220, and/or a dedicated image processing unit or pipeline incorporated within circuitry 265. Raw sensory data so captured may be stored in memory 260 and/or storage 265.

Memory 260 may include one or more different types of media used by processor 205 and graphics hardware 220 to perform device functions. For example, memory 260 may include memory cache, read-only memory (ROM), and/or random-access memory (RAM). Storage 265 may store media (e.g., audio, image and video files), computer program instructions or software, preference information, device profile information, and any other suitable data. Storage 265 may include one more non-transitory computer-readable storage mediums including, for example, magnetic disks (fixed, floppy, and removable) and tape, optical media such as CD-ROMs and digital video disks (DVDs), and semiconductor memory devices such as Electrically Programmable Read-Only Memory (EPROM), and Electrically Erasable Programmable Read-Only Memory (EEPROM). Memory 260 and storage 265 may be used to tangibly retain computer program instructions or code organized into one or more modules and written in any desired computer programming language. When executed by, for example, processor 205 such computer program code may implement one or more of the methods described herein.

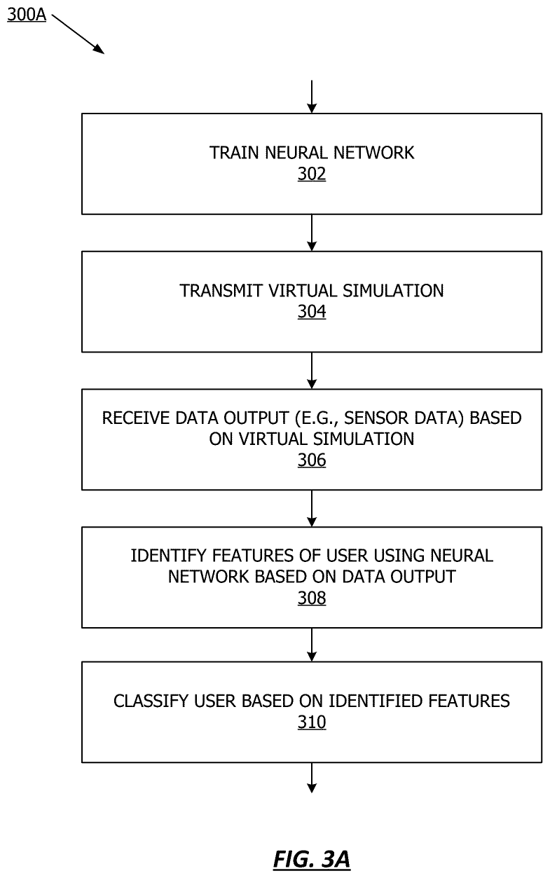

FIG. 3A shows, in flow chart form, an example method for performing a VRNA assessment in accordance with an embodiment of the disclosure. The method may be implemented by module 118 of FIG. 1. For purposes of explanation, the following will be described in the context of FIG. 1. However, it should be understood that the various actions may be taken by alternate components. In addition, the various actions may be performed in a different order. Further, some actions may be performed simultaneously, and some may not be required, or others may be added.

The flow chart begins at 302 where VRNA service 110 trains a neural network. The neural network may be, for example, module 118 on memory 114. As described herein, the neural network may be trained using a plurality of training datasets. According to one or more embodiments, training datasets may be obtained from a user of VRNA service 110, from a remote device, such as client device 130, or from a third party.

The flow chart continues at 304 where VRNA service 110 transmits a virtual simulation to client device 130. The virtual simulation, as described herein, may be a computer program that simulates a combat-like scenario. While a combat-like scenario is provided to illustrate the disclosed technology, it is understood that users may participate in other interactive game-like decision-making scenarios without departing from the scope of the disclosed. The computer program may provide, during the combat-like scenario, a multitude of tests intended to evaluate a user's reaction time, decision-making, or the like. The user may experience the virtual simulation using a VR headset that provides an immersive experience. Additionally, the VR headset may include a plurality of sensors to collect data during the virtual simulation. Sensors may be provided as part of the VR headset, attached to the user (e.g., wrist-worn sensors, HR monitor worn around the chest), or a combination thereof. The data collected may include the user's heartrate, blood pressure, eye gaze data, or the like.

The flow chart continues at 306 where VRNA service 110 receives data from client device 130. Data received from client device 130 may include results from the virtual simulation. The results may include the above-mentioned sensor output (e.g., user's vitals) collected during the virtual simulation. Additionally, results of the virtual simulation with respect to the user's performance may be provided. Results may include, for example, confirmed targets, missed targets, pass/fail metrics for missions, or the like.

The flow chart continues at 308 where VRNA service 110 identifies features that pertain to the user. Features may be identified using the neural network of module 118. Results obtained in 306, such as the sensor data output and user performance during the virtual simulation may be provided as input to module 118.

The flow chart continues at 310 where VRNA service 110 classifies the user based on the identified features of the user. A user may be classified using the neural network of module 118.

FIG. 3B shows, in flow chart form, an example method for conducting a VRNA assessment in accordance with an embodiment of the disclosure. The method may be implemented by module 138 of FIG. 1. For purposes of explanation, the following will be described in the context of FIG. 1. However, it should be understood that the various actions may be taken by alternate components. In addition, the various actions may be performed in a different order. Further, some actions may be performed simultaneously, and some may not be required, or others may be added.

The flow chart begins at 320 where client device 130 receives a virtual simulation. The virtual simulation may be a video game played in virtual reality using special hardware. VR hardware may include, for example, a VR headset that provides players with an immersive experience. The VR headset may include a head-mounted display that has a stereoscopic display. The VR headset may also be connected to one or more input devices, such as a keyboard, a mouse, hand-held controllers, positional tracking devices, or combinations thereof.

The flow chart continues at 322 where client device 130 runs the virtual simulation. The virtual simulation may be provided, as described above, using a VR headset and one or more accessories, such as VR headset and accessories 150 of FIG. 1. During the virtual simulation, sensor data may be collected by sensors of the headset or sensors attached to the headset, either wired or wirelessly. For example, a heart rate monitor may provide heart rate information during the virtual simulation over a short-range wireless connection to the headset or the client device 130 in real-time. Collected data may be assigned metadata, such as timestamp information.

The flow chart continues at 324 where client device 130 receives data output of the virtual simulation. Data output may be received from a plurality of different sources (e.g., VR headset, wearable sensors) during the virtual simulation. For example, sensor data may be captured by a plurality of sensors of VR headset 150. Additionally, sensor data may be captured by one or more accessories of VR headset 150, such as a hand-held controller or a heart monitor, during the virtual simulation.

The flow chart continues at 326 where client device 130 continues to receive data during the virtual simulation. At 326, client device 130 organizes all the collected data and the flow chart continues at 328 where the collected data is transmitted to an assessment module, such as module 118 described herein.

### Conceptual Overview of the Virtual Reality Neuropsychological Assessment (VRNA)

To assess real-time neuropsychological performance, an assessment may be provided using a virtual scenario. For example, the virtual scenario may be a 3D simulation of combat activity involving game-like decision-making scenarios presented via a VR headset and hand controllers. While a combat-like simulation is provided to illustrate the disclosed technology, it is understood that users may participate in other interactive game-like decision-making simulations without departing from the scope of the disclosed. In some embodiments, participants may wear the VR headset to enter a virtual combat scenario where they conduct a predefined mission while engaging enemy combatants, friendly forces, non-combatants, and various aerial and land vehicles. Rules of engagement (i.e., contingencies) would define when to fire or hold-fire on combatants based on stimulus characteristics and the situational context. Parameters of the stimuli may be parametrically modulated to require varying levels of cognitive performance. By parametrically modulating the cognitive load (i.e., task difficulty), several dimensions of cognitive performance may be extracted simultaneously.

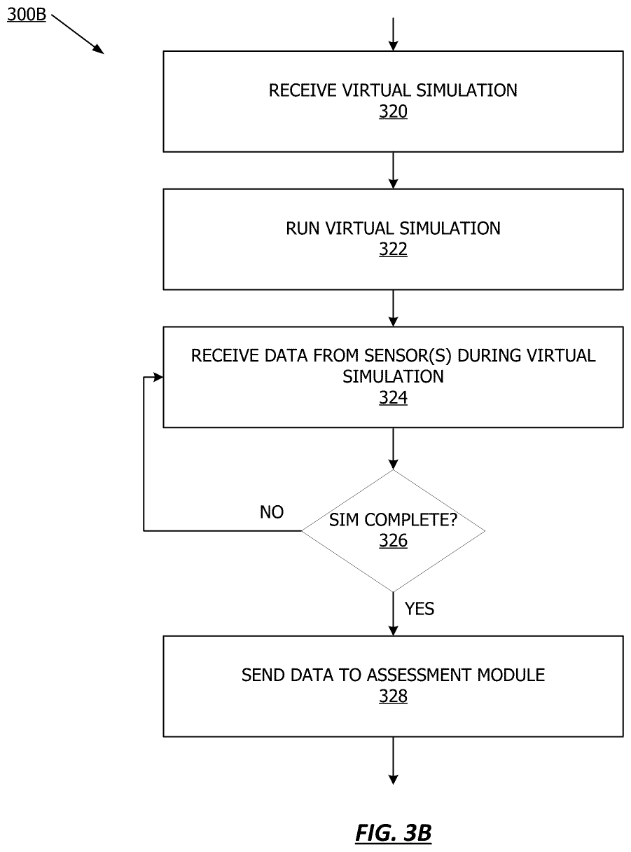

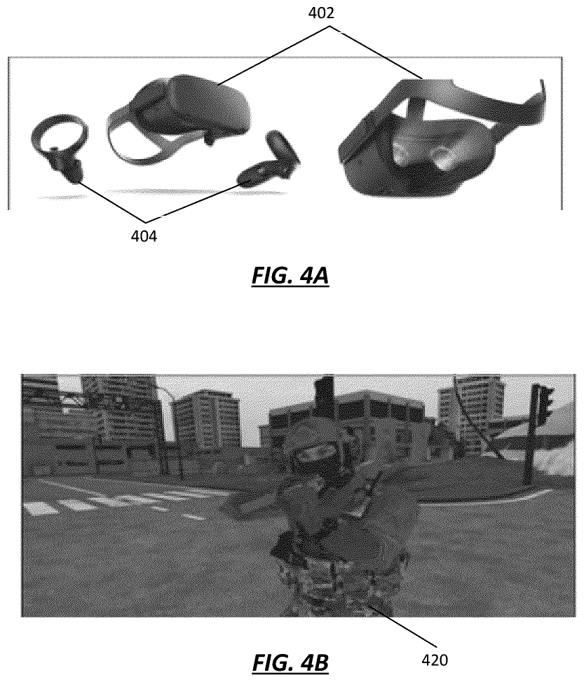

In some embodiments, four domains most likely to discriminate between mTBI and healthy participants may be scored. The four domains may include, for example, attention, processing speed, learning/memory, and executive control. Stimulus presentation and data collection may be performed using a VR headset as described herein that provides an immersive experience. In one example, the VR headset may include a plurality of cameras (e.g., five cameras) along with an open-source library for program development in 3D. FIG. 4A shows an example headset 402 with controllers 404. FIG. 4B illustrates an example avatar 420 for use in the virtual simulation. In this example, the avatar is a soldier avatar created in 3D via the open-source library. Avatars may be provided via an application interface library. Users may be provided with the ability to create their own avatars within the virtual simulation. In some embodiments, avatars may be created using an established set of customization options. For example, customization options may include the development of military combatants and civilian personnel within a virtual simulation of a combat-like scenario. Avatars may also provide hand presence for controllers, such as controllers 404. Hand presence for the controllers may be used to integrate VR headset interaction with inputs used for assessment (e.g., as data inputs to neural network to identify and classify users). The VR headset may include a comprehensive set of application program interfaces that allows direct communication with the headset and controller in real time. Additionally, or alternatively, the application program interfaces enable the creation of background scenes (e.g., 3D military background scenes) and stimuli with which the participant will interact during the virtual simulation. That provides an ecologically valid approach to assessing militarily relevant performance domains that are more aligned with actual tasks (e.g., speed of movement, visually locating targets, firing or holding fire based on the circumstances) than legacy neuropsychological tests.

### Deep Learning Neural Network (DNN)

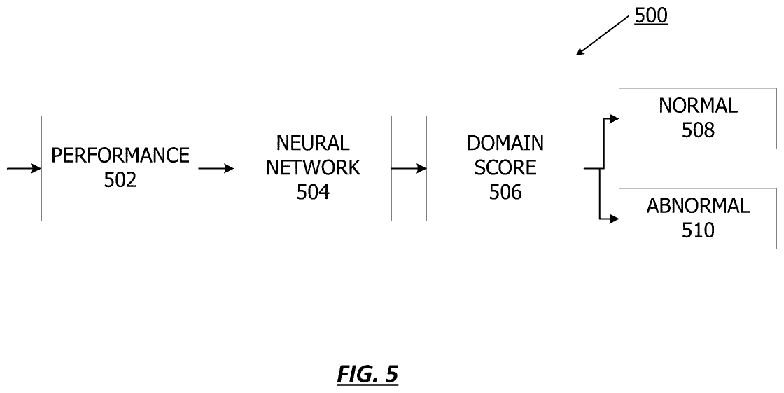

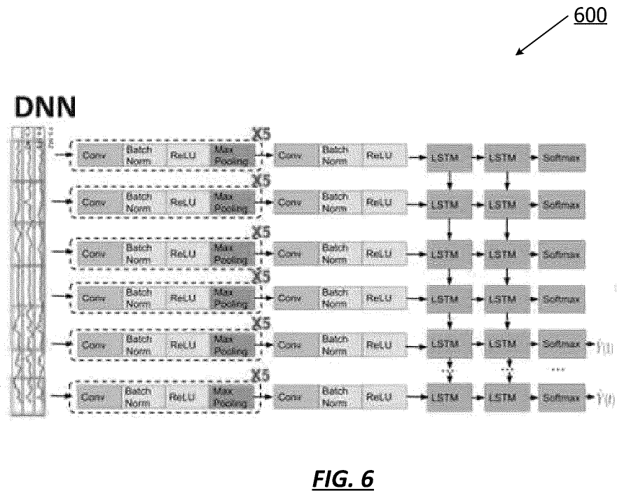

In accordance with embodiments described herein, a Deep Neural Network (DNN) is provided by the disclosed technology. An example DNN network 600 described herein is shown in FIG. 6. An example flow chart 500 of a VRNA assessment is shown in FIG. 5. In one example, raw sensory data linked to the participant's activities 502 during the virtual simulation may be provided 504 as input to the DNN. Raw sensory data may include, but is not limited to, reaction time, processing speed, working memory, and executive function. Additionally, the DNN may provide multi-dimensional performance assessment in real time. The DNN may employ long short memory and convolution techniques to incorporate temporal and spatial correlations into one network, as illustrated. As illustrated in FIG. 6, Long short-term memory (LSTM) networks may be used on the training dataset, incorporating eye-tracking, hand-eye motor coordination, reaction time, working memory, learning and delayed memory, and inhibitory control to create a sequence of temporal and spatial correlations in the DNN. LSTM is a special type of recurrent neural network using “memory cells” to remember information from previous LSTM units for learning long-term dependencies and is suitable for the disclosed as it helps to bridge the difference between timescales. The LSTM unit may include two layers. In some embodiments, two recurrent layers are beneficial when processing sequential data. The resultant models reveal how each type of raw data corresponds to the final performance assessment score. Furthermore, a CNN-LSTM model may be used. For example, convolutional neural networks (CNNs) and LSTMs may be implemented for the sequence-to-sequence learning task. CNN is a class of deep artificial neural network which can be used to extract features from signals. As shown in FIG. 6, it combines multiple filters with a moving window. Comparing with other LSTM models, this CNN-LSTM model does not use hand-engineered features. In contrast, it uses learned features by the CNN. The CNN-LSTM model takes raw data as inputs, where the outputs are performance assessment results (i.e., score or index) in 506. The assessment results may be used to determine whether a participant has normal (e.g., healthy) 508 or abnormal (e.g., mBTI) 510 characteristics. In some embodiments, the DNN may converge on a best set of weights embedded on edges among layers of the neural network to mimic the domains measured by the traditional neuropsychological test battery. In some embodiments, the DNN may be trained using patients with mTBI versus healthy controls. Upon completion of the DNN training, the VRNA system may provide output metrics for the four primary neurocognitive domains used to discriminate mTBI from HCs (e.g., Attention, Processing Speed, Working Memory, and Executive Function). In some embodiments, the trained DNN may be downloaded to each headset to provide real-time performance assessment in an independent validation sample.

### VR Environment Development

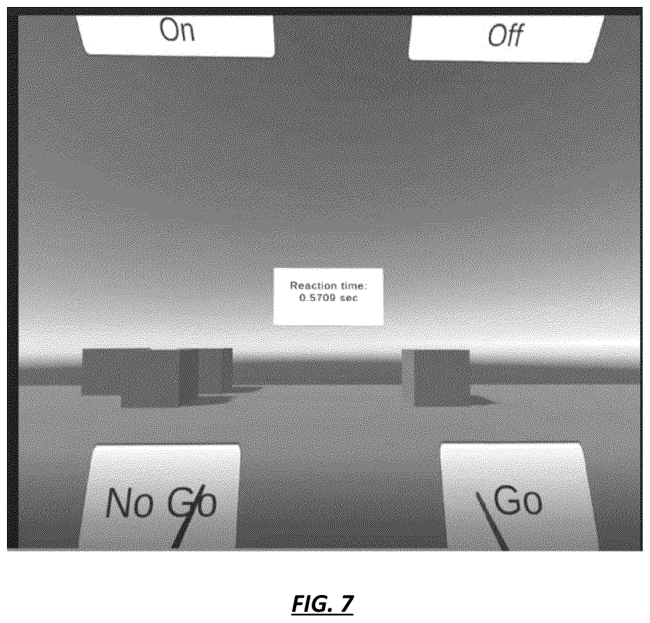

FIG. 7 illustrates an example gaming environment and presentation in accordance with at least one embodiment of the disclosed technology. As shown, a VR game environment of a simple go/no-go type system represented in a military game. In the example, a model “shoot/no-shoot” task is illustrated to test a player's reaction time. In this example, a “soldier” spawner and a reaction timer are implemented. Cubes are provided as placeholders and the game mechanic included basic go/no-go logic. In different embodiments, different conditions may be set to test the user, such as environments (e.g., urban or rural), as well as contingencies (e.g. tan cubes in an urban environment or green in a rural environment is “go,” while green in an urban environment or tan in a rural environment is “no-go”). In some embodiments, the contingencies may be reversed during the assessment. The implementation may include audio sound effects and history data export features. History data export features allow for the tracking of settings, accuracy, reaction time, time-stamp history, or the like.

### Implementation Example

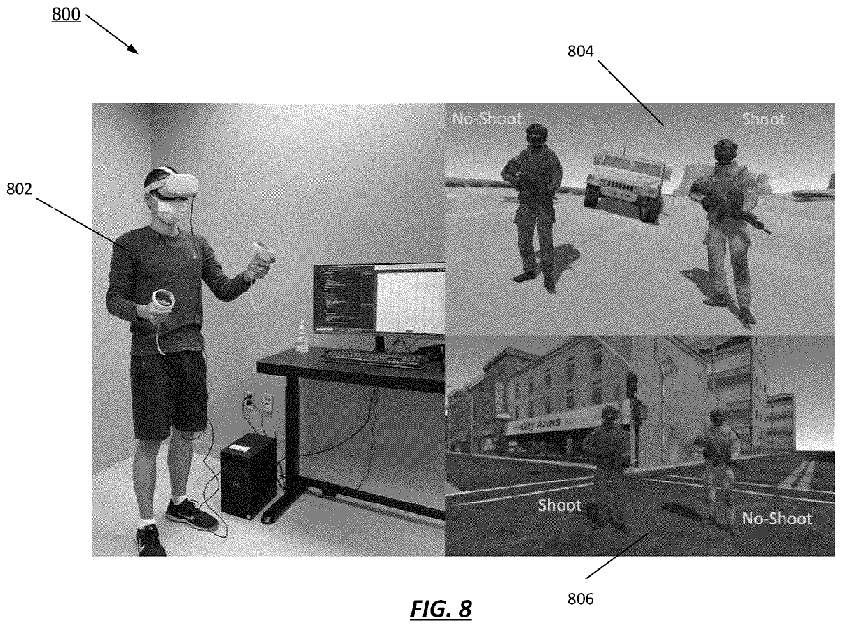

In view of the example illustration 800 of FIG. 8, an example implementation of a VRNA assessment is provided. In this example, a participant 802 may wear a VR headset and use hand controllers, such as VR Headset and Accessories 150 described in FIG. 1. Modifications may be provided due to future technological advances without departing from the scope of the disclosed. Upon donning the VR headset, the participant may be provided with a short briefing on the “mission.” The participant may then enter a virtual urban combat scenario where they must carry out a predefined mission while engaging enemy combatants, friendly forces, non-combatants, and various aerial and land vehicles, see virtual screens 804 and 806. Rules of engagement (i.e., contingencies) may define when to fire or hold-fire on combatants based on stimulus characteristics and the situational context. As seen in virtual screen 804, when in a rural environment, the participants must shoot (i.e., “go”) characters wearing green (“No-Shoot” character on left), but hold fire (i.e., “no-go”) to characters wearing tan uniforms (“Shoot” character on right), while the opposite rules apply in urban scenario. To assess cognitive flexibility, these contingencies may be reversed halfway through the session. The provided example is intentionally simple in design for ease of explanation. It is understood that virtual simulations used for a VRNA assessment may significantly increase in complexity of stimuli and scenarios. The parameters of the stimuli may be parametrically modulated to require varying levels of attention, real-time learning and memory, executive function, and rapid processing speed. By parametrically modulating the cognitive load (i.e., task difficulty) on these domains, it may be possible to extract several dimensions of cognitive performance simultaneously. In some versions, the VRNA assessment may be focused on a limited set of domains-which are likely to discriminate between mTBI and healthy participants (i.e., attention, processing speed, learning/memory, and executive control). In some embodiments of the VRNA assessment may focus on additional neurocognitive domains or a subset thereof. Additional neurocognitive domains may include, for example, written and auditory language comprehension, speech fluency assessment, eye-tracking/oculomotor, visuospatial abilities, motor control, vestibular/balance, and coordination. In some embodiments, greater nuance may be given to executive functioning/problem solving, working memory, and other domains.

### Machine Learning Program Development

In some embodiments of the disclosed technology, the VRNA assessment may include one or more machine learning (ML) algorithms. The one or more ML algorithms may be based on the programmed “shoot/no-shoot” task described herein as a basis for developing the core program concept that would align with the VR model. Additionally, the one or more ML algorithms may be based on certain datasets to develop machine learning models that could be compared for predictive accuracy.

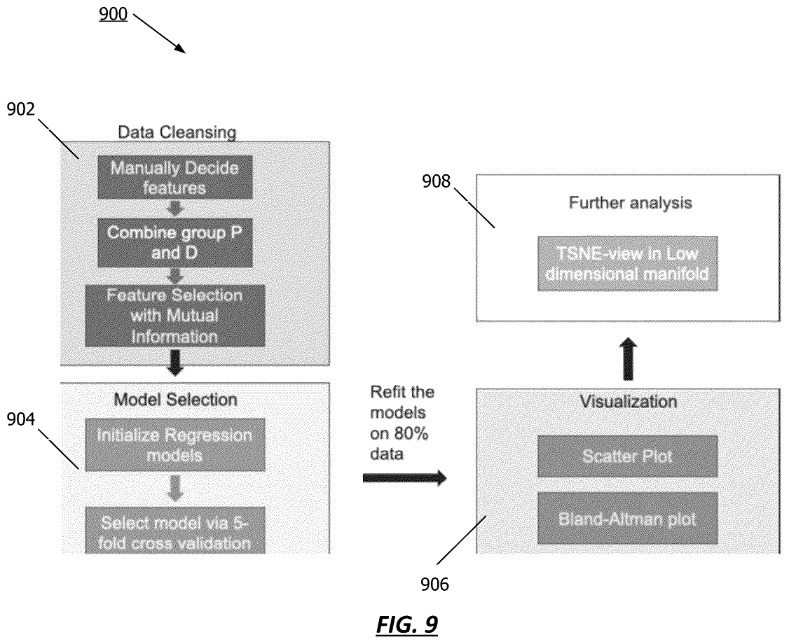

In an example embodiment of a VRNA assessment module, preliminary data from a Context Dependent Shoot/No-Shoot (SNS) Task may be used. Data was made available from 359 participants (mTBI n=191; healthy control-HC n=120; sleep deprived-SD n=48) who completed the SNS, psychomotor vigilance test (PVT), and various neurocognitive tasks. As a first step, the data were used to build a regression pipeline to predict simple reaction time on the PVT. FIG. 9 shows an example overall analysis flow 900 from data cleansing 902 to model selection 904, to visualization 906 to further analysis 908. Various models may be tested including multi-layer perception regression (artificial neural network), Bayesian regression, linear regression, support vector regressor, XGBoost regressor, and Kernel Ridge regression. Each model may be evaluated using five-fold cross-validation. First, in 902, training and validation datasets may be constructed using the demographic information (age and gender), the WAST test scores, and the SNS scores as the inputs and using the PVT as the learning target. For the HC group, select the baseline day's SNS scores and reaction time as inputs and learning targets, respectively. For the SD group, choose the second day's (i.e., 24 hours of sleep deprivation) SNS scores and reaction time as inputs and learning targets, respectively. The correlation between the reaction time and inputs may be analysed by mutual information feature selection. The most important features are selected 904 to build the six regression models. For example, multi-layer perception regression (artificial neural network), Bayesian regression, linear regression, support vector regressor, XGBoost regressor, and Kernel Ridge regression. The models may be refit on 80% of the data to visualize the difference between prediction results and true targets by a Pearson's correlation plot and a Bland-Altman plot. Both a 3D and 2D t-distributed stochastic neighbor embedding (tSNE) view of the data on a lower-dimensional manifold may be included to evaluate the quality of the dataset. The models may be trained and evaluated by five-fold cross-validation.

In some embodiments of the disclosed technology, employing the full DNN approach may require extensive data collection from participants who use the VR system and concomitantly take a full battery of neurocognitive assessments. One objective is to identify multivariate predictors from the VR game scenario that track closely to the primary domain scores from the traditional neuropsychological assessment battery and that discriminate between healthy individuals and those with various neurological conditions.

According to some embodiments, a processor or a processing element may be trained using supervised machine learning and/or unsupervised machine learning, and the machine learning may employ an artificial neural network, which, for example, may be a convolutional neural network, a recurrent neural network, a deep learning neural network, a reinforcement learning module or program, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

According to certain embodiments, machine learning programs may be trained by inputting sample data sets or certain data into the programs, such as images, object statistics and information, historical estimates, and/or image/video/audio classification data. The machine learning programs may utilize deep learning algorithms that may be primarily focused on pattern recognition and may be trained after processing multiple examples. The machine learning programs may include Bayesian Program Learning (BPL), voice recognition and synthesis, image or object recognition, optical character recognition, and/or natural language processing. The machine learning programs may also include natural language processing, semantic analysis, automatic reasoning, and/or other types of machine learning.

According to some embodiments, supervised machine learning techniques and/or unsupervised machine learning techniques may be used. In supervised machine learning, a processing element may be provided with example inputs and their associated outputs and may seek to discover a general rule that maps inputs to outputs, so that when subsequent novel inputs are provided the processing element may based upon the discovered rule, accurately predict the correct output. In unsupervised machine learning, the processing element may need to find its own structure in unlabeled example inputs.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

What is claimed is:

1. A virtual reality neuropsychological assessment (VRNA) system, comprising:
at least one non-transitory computer readable medium comprising:
a deep learning network trained to i) identify features in sensor data indicative of neuropsychological performance and ii) classify users based on the features identified in the sensor data;
a processor configured to:
transmit, to a client device, a virtual simulation of an activity involving decision-making scenarios;
receive, from the client device, captured sensor data;
identify, using the deep learning network, one or more features of a user of the client device based on the captured sensor data; and
classify, using the deep learning network, the user based on the one or more features.

2. The VRNA system of claim 1, wherein the activity is a virtual combat scenario.

3. The VRNA system of claim 1, wherein the deep learning network is trained to:
calculate scores for the users, based on the features identified in the sensor data, in a plurality of neuropsychological domains; and
classify the users based on the calculated scores in each of the plurality of neuropsychological domains.

4. The VRNA system of claim 3, wherein the plurality of neuropsychological domains comprise one or more of attention, memory, processing speed, and executive function.

5. The VRNA system of claim 1, wherein the sensor data comprises eye-tracking data and data indictive of hand-eye motor coordination, reaction time, working memory, learning and delayed memory, and inhibitory control.

6. The VRNA system of claim 1, wherein the deep learning network comprises:
a long short-term memory (LSTM) network that uses training data to create a sequence of temporal and spatial correlations in the deep learning network; and
a convolutional neural network (CNN) trained using the training data to extract features from the captured sensor data.

7. A virtual reality neuropsychological assessment (VRNA) system, comprising:
at least one non-transitory computer readable medium comprising:
a deep learning network trained to i) identify features in sensor data indicative of neuropsychological performance and ii) classify users based on the features identified in the sensor data;
a processor configured to:
transmit, to a client device, a virtual simulation of an activity involving decision-making scenarios;
receive, from the client device, captured sensor data;
identify, using the deep learning network, one or more features of a user of the client device based on the captured sensor data; and
classify, using the deep learning network, the user based on the one or more features.

8. The VRNA system of claim 7, wherein the activity is a virtual combat scenario.

9. The VRNA system of claim 7, wherein the deep learning network is trained to:
calculate scores for the users, based on the features identified in the sensor data, in a plurality of neuropsychological domains; and
classify the users based on the calculated scores in each of the plurality of neuropsychological domains.

10. The VRNA system of claim 9, wherein the plurality of neuropsychological domains comprise one or more of attention, memory, processing speed, and executive function.

11. The VRNA system of claim 7, wherein the sensor data comprises eye-tracking data and data indictive of hand-eye motor coordination, reaction time, working memory, learning and delayed memory, and inhibitory control.

12. The VRNA system of claim 7, wherein the deep learning network comprises:
a long short-term memory (LSTM) network that uses training data to create a sequence of temporal and spatial correlations in the deep learning network; and
a convolutional neural network (CNN) trained using the training data to extract features from the captured sensor data.

13. The VRNA system of claim 7, wherein the client device provides the virtual simulation to the user wearing a head-mounted device having a stereoscopic display.

14. The VRNA system of claim 13, wherein the sensor data is captured by one or more sensors connected to the head-mounted device.

15. A method for providing a virtual reality neuropsychological assessment (VRNA), comprising:
training a deep learning network to i) identify features in sensor data indicative of neuropsychological performance and ii) classify users based on the features identified in the sensor data;
transmitting, to a client device, a virtual simulation of an activity involving decision-making scenarios;
receiving, from the client device, captured sensor data;
identifying, using the deep learning network, one or more features of a user of the client device based on the captured sensor data; and
classifying, using the deep learning network, the user based on the one or more features.

16. The method of claim 15, wherein the activity is a virtual, interactive activity involving decision-making scenarios.

17. The method of claim 15, further comprising:
training the deep learning network to calculate scores for the users, based on the features identified in the sensor data, in a plurality of neuropsychological domains; and
classifying the users based on the calculated scores in each of the plurality of neuropsychological domains.

18. The method of claim 17, wherein the plurality of neuropsychological domains comprise one or more of attention, memory, processing speed, and executive function.

19. The method of claim 15, wherein the sensor data comprises eye-tracking data and data indictive of hand-eye motor coordination, reaction time, working memory, learning and delayed memory, and inhibitory control.

20. The method of claim 15, wherein the deep learning network comprises:
a long short-term memory (LSTM) network that uses training data to create a sequence of temporal and spatial correlations in the deep learning network; and
a convolutional neural network (CNN) trained using the training data to extract features from the captured sensor data.
